(19) United States
(12) Patent Application Publication
(10) Pub. No.: US 2026/0168947 A1
(43) Pub. Date: Jun. 18, 2026

(54) SENSOR DEVICE BASED ON GALLIUM OXIDE AND MANUFACTURING METHOD THEREOF, IN PARTICULAR FOR GAS SENSING

(71) Applicant: STMicroelectronics International N.V., Geneva (CH)

(72) Inventors: Delfo Nunziato SANFILIPPO, Catania (IT); Davide Giuseppe PATTI, Mascalucia (CT) (IT); Nadia LICCIARDELLO, Acireale (IT)

(73) Assignee: STMicroelectronics International N.V., Geneva (CH)

(21) Appl. No.: 19/416,185

(22) Filed: Dec. 11, 2025

(30) Foreign Application Priority Data
Dec. 13, 2024 (IT) 102024000028401

(51) Int. Cl. G01N 27/12; G01N 33/00; H10D 30/65; H10D 62/832

(52) U.S. Cl. CPC G01N 27/125; G01N 33/0027; H10D 30/65; H10D 62/8325

(57) ABSTRACT

A sensor device includes a substrate made of 4H-SiC polytype silicon carbide having a first electrical conductivity type and a first sensitive region of semiconductor material on a first side of the substrate. A first conductive region is located on and in electrical contact with the first sensitive region. The first sensitive region is made of β-polymorph crystalline gallium oxide (β-Ga₂O₃) having the first electrical conductivity type.

### PRIORITY CLAIM

This application claims the priority benefit of Italian Application for Patent No. 102024000028401 filed on Dec. 13, 2024, the content of which is hereby incorporated by reference in its entirety to the maximum extent allowable by law.

### TECHNICAL FIELD

The present invention relates to a sensor device, to the manufacturing method thereof and to a die including the sensor device. In particular, the sensor device is configured to operate as a gas sensor.

### BACKGROUND

Given the increasing global atmospheric pollution, there is an increasing need to sense polluting or toxic gases present in the air. This need may also be found in other fields, for example in the industrial field where the presence, prolonged for a given time, of (even low) amounts of toxic or polluting gases may cause health problems. This need may also be found in situations where the leakage of dangerous gases may cause fires, explosions or other types of adverse events.

Therefore, sensing the presence of gases in different types of environments may be advantageous to prevent the aforementioned issues.

There is therefore a need to provide a method for manufacturing a sensor, and the sensor thereof, that are adaptable to various applications and environments (including harsh environments), inexpensive and efficient.

### SUMMARY

The present invention relates to a sensor device, to the manufacturing method thereof and to a die including the sensor device.

In an embodiment, a sensor device comprises: a substrate of 4H-SiC polytype silicon carbide, having a first electrical conductivity; a first sensitive region of semiconductor material on one side of the substrate; and a first conductive region on the first sensitive region, in electrical contact with the first sensitive region, wherein the first sensitive region is of β-polymorph crystalline gallium oxide, β-Ga₂O₃, having the first electrical conductivity. The device further comprises an insulating layer between the first and the second sensitive regions, where the insulating layer is made of one of: silicon oxide, silicon nitride, TEOS, undoped β-Ga₂O₃.

In an embodiment, a semiconductor die comprises the sensor device according to the foregoing and: a transistor having a gate terminal, a first conduction terminal and a second conduction terminal, at least in part integrated in the substrate, the gate terminal being electrically coupled to the first conductive region; a first resistance coupled between a first bias terminal and a first electrical node common to the gate terminal and the first conductive region; and a second resistance coupled between a second bias terminal and one of the first and the second conduction terminals, wherein the other of the first and the second conduction terminals is electrically coupled to the first bias terminal.

In an embodiment, a method for manufacturing a sensor device, in particular a gas sensor, comprises the steps of: forming, on one side of a substrate of 4H-SiC polytype silicon carbide, having a first electrical conductivity, a first sensitive region of semiconductor material having the first electrical conductivity; and forming a first conductive region on the first sensitive region, in electrical contact with the first sensitive region; wherein the step of forming the first sensitive region comprises performing a Metal-Organic Chemical Vapor Deposition, MOCVD, growth process of β-polymorph crystalline gallium oxide, β-Ga₂O₃. The first sensitive region may be isolated from other sensitive regions by an insulating layer made of one of: silicon oxide, silicon nitride, TEOS, undoped β-Ga₂O₃.

### BRIEF DESCRIPTION OF THE DRAWINGS

For a better understanding of the present invention, some embodiments thereof are now described, purely by way of non-limiting example, with reference to the attached drawings, wherein:

FIGS. 1A-1I illustrate manufacturing steps of a sensor device;

FIG. 2 illustrates a sensor device;

FIG. 3 illustrates a gas sensor; and

FIG. 4 illustrates a gas sensor.

### DETAILED DESCRIPTION

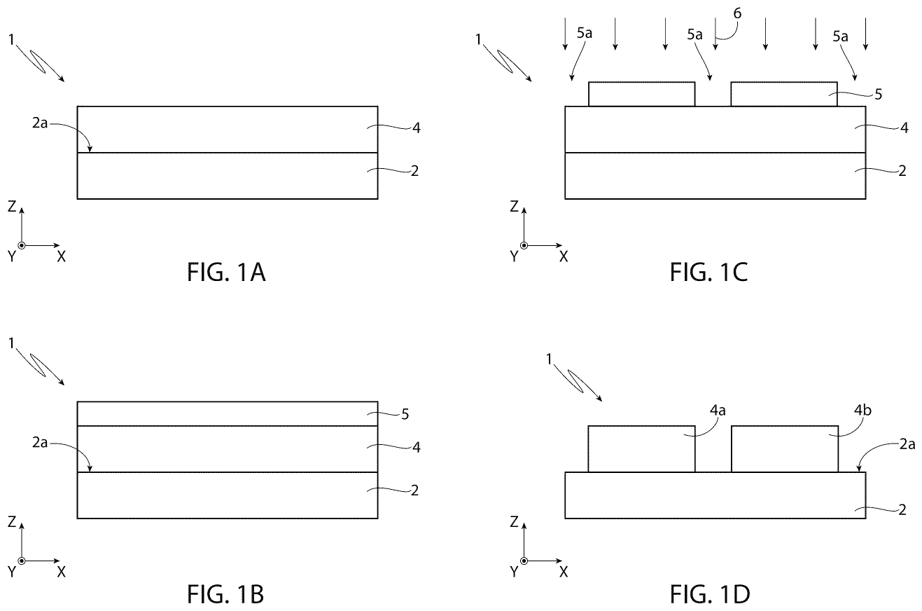

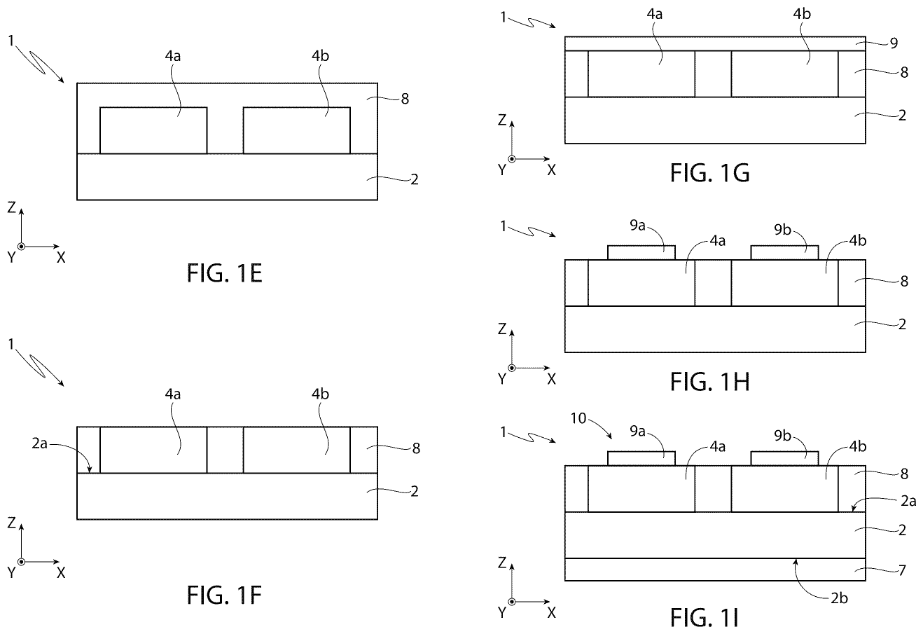

FIGS. 1A-1I illustrate, in a lateral sectional view in a triaxial reference system of axes X, Y, Z orthogonal to each other, steps of a manufacturing process of a sensor device 10. In particular, the sensor device 10 is a gas sensor. FIGS. 1A-1I illustrate processing steps of a wafer, or die, 1 limitedly to a portion of the same of interest for embodiments herein.

With reference to FIG. 1A, a substrate 2 made of silicon carbide (SiC), in particular 4H-SiC, is deposited, having an N-type electrical conductivity. The substrate 2 has doping for example comprised between 10¹⁶ and 10¹⁹ at/cm³.

Alternatively, the electrical conductivity of the substrate 2 may be of the P-type.

The substrate 2 may comprise, in the context of the embodiments, one or more epitaxial layers, as needed, and has a thickness, along the Z-axis, comprised between 50 μm and 500 μm.

A sensor layer 4, in particular made of an ultra-wide bandgap semiconductor, even more in particular made of gallium oxide (Ga₂O₃), is formed on a surface 2a of the substrate 2. Ga₂O₃ comes in different polytypes; the polytype of interest for the embodiments is the beta (β) phase, which is thermodynamically stable. Therefore, in the context of the embodiments, the sensor layer 4 is made of β-Ga₂O₃. In particular, the sensor layer 4 is made of crystalline β-Ga₂O₃.

The formation of the sensor layer 4 occurs, for example, by Metal-Organic Chemical Vapor Deposition (MOCVD), which is known per se.

In one embodiment, the material of the sensor layer 4 is deposited concurrently with doping agents, so as to form the sensor layer 4 having the desired electrical conductivity. For example, an N-type doping is obtained by doping with silicon (Si) or tin (Sn). In a further embodiment, the sensor layer 4 is deposited in the absence of doping agents, to form the undoped sensor layer 4. Following the deposition, an implant step of doping species having the desired electrical conductivity (e.g., of the N-type) is performed, to obtain the doped sensor layer 4.

For example, in both embodiments, the doped sensor layer 4 has a dopant concentration comprised in the range 10¹⁶-10¹⁹ at/cm³ (range boundaries included).

When doped, the sensor layer 4 has the characteristics of a semiconductor material; when undoped, the sensor layer 4 has the characteristics of an insulating material.

By way of non-limiting example, a MOCVD growth process of the sensor layer 4 made of β-Ga₂O₃ on the substrate 2 of 4H-SiC is described hereinbelow. Typically, β-Ga₂O₃ on SiC is grown in a temperature range between 600° C. and 950° C., at pressures between 50 and 200 mbar, with gallium and oxygen precursors and carrier gases. For example, a temperature of 800° C., a pressure of 100 mbar, trimethylgallium (TMG) and oxygen as precursors and argon as carrier gas may be used for the MOCVD growth of the sensor layer 4 of β-Ga₂O₃ on the substrate 2 of 4H-SiC.

Then, with reference to FIG. 1B, a mask layer 5 is formed on the sensor layer 4. For example, the mask layer 5 is made of silicon oxide (SiO₂) deposited by CVD technique. It is evident that the mask layer 5 may be made of other materials, or formed by other deposition techniques. For example, alternative materials for the mask layer 5 include silicon nitride and tetraethyl orthosilicate (TEOS).

Then, as shown in FIG. 1C, the mask layer 5 is patterned so as to completely remove portions of the mask layer 5 at portions of the sensor layer 4 that are desired to be removed. The mask layer 5 is patterned so as to define openings 5a having, in top view on the XY plane, the shape of strips with the main dimension along the Y-axis. Other shapes of the openings 5a are possible, for example generally polygonal or curvilinear shapes.

The mask layer 5, therefore, remains above selective portions of the sensor layer 4 that are desired to be maintained unchanged. An etch step of the sensor layer 4 is then performed, for example a plasma dry etching (identified in FIG. 1C by arrows 6) using boron trichloride (BCl₃) as a chemical agent to remove the portions of the sensor layer 4 not protected by the mask 5.

The exposed (unmasked) portions of the sensor layer 4 are removed throughout their entire thickness (along the Z-axis), until reaching and exposing respective portions of the surface 2a of the substrate 2 (FIG. 1D). Optionally, a further etch step of the sensor layer 4 is performed, using a wet etching based on phosphoric acid (H₃PO₄) at a temperature comprised between about 120 and 150° C. (e.g., 140° C.). This further etching has the function of removing any β-Ga₂O₃ residues not removed by the preceding dry etching. The mask 5 is then removed.

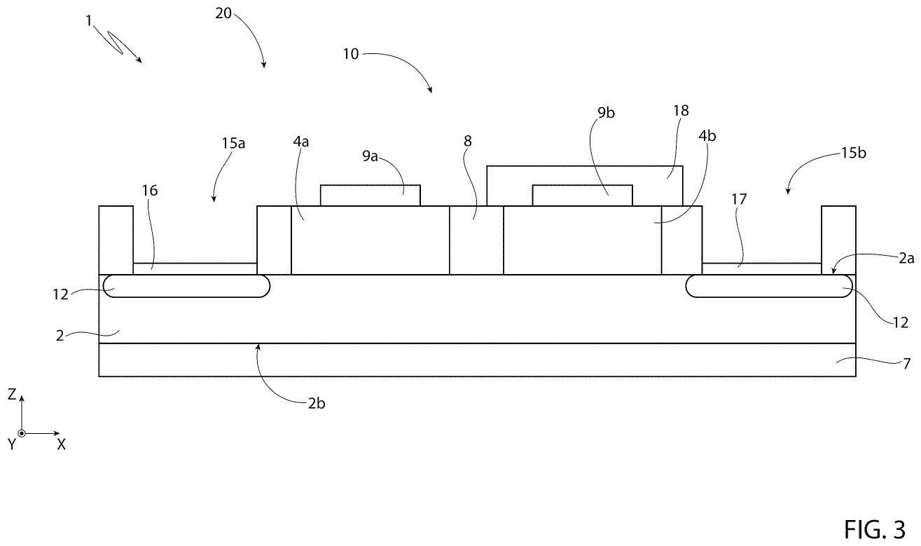

One or more sensor elements are thus formed (two sensor elements 4a, 4b are illustrated in the Figure, without thereby losing generality). In particular, as may be better appreciated from the description of FIG. 3, the use of two sensor elements 4a, 4b allows one sensor element (e.g., 4b) to be used as a reference and the other sensor element (e.g., 4a) as a sensitive element.

Subsequently, as shown in FIG. 1E, an insulation layer 8 is formed, for example made of silicon oxide (SiO₂), or silicon nitride (SiN), or TEOS, or other insulating material, above the sensor elements 4a, 4b and laterally to the sensor elements 4a, 4b, in particular between the sensor element 4a and the sensor element 4b.

With reference to FIG. 1F, the insulation layer 8 is then removed above the sensor elements 4a, 4b (until exposing the surface of the sensor elements 4a, 4b), and remains between the sensor element 4a and the sensor element 4b. Furthermore, the insulation layer 8 is also maintained laterally to both the sensor elements 4a and 4b. The step of FIG. 1F is performed, for example, by a chemical-mechanical planarization (CMP) technique.

Then, as shown in FIG. 1G, a conductive layer 9, in particular a metal layer, is deposited above the sensor elements 4a, 4b and the insulation layer 8. In particular, the conductive layer 9 is in direct physical and electrical contact with the sensor elements 4a, 4b. The material of the conductive layer 9 is chosen as a function of the application of the sensor device 10. For example, a conductive layer 9 made of Platinum (Pt) allows a Schottky contact to be formed with the underlying sensor elements 4a, 4b. A conductive layer 9 made of Titanium (Ti) allows an ohmic contact to be formed with the underlying sensor elements 4a, 4b.

Other materials for the conductive layer 9 comprise, for example, Palladium (Pd), Nickel

### (ni) and Tungsten (w).

Then, as shown in nFigure 1H, the conductive layer 9 is patterned (e.g., by photolithography techniques) so as to remove selective portions of the conductive layer 9 that extend above the insulation layer 8, forming electrical contact regions 9a, 9b, electrically insulated from each other. The electrical contact region 9a extends exclusively above, and in contact with, the sensor element 4a; the electrical contact region 9b extends exclusively above, and in contact with, the sensor element 4b. The electrical contact regions 9a, 9b may cover the surface of the sensor elements 4a, 4b completely or only in part.

It is evident that the electrical contact regions 9a, 9b may, in other embodiments, also extend above the insulation layer 8, while remaining electrically insulated from each other.

Furthermore, as shown in FIG. 1I, an electrical contact 7 is formed on the back of the substrate 2, i.e., at a surface 2b of the substrate 2, opposite to the surface 2a along the Z-axis. The electrical contact 7 is in electrical connection with the substrate 2, directly or through an intermediate layer (for example an ohmic contact).

Optionally, a protective layer (not illustrated) that covers in whole or in part one or both of the electrical contact regions 9a, 9b may be formed; such a protective layer has, for example, the functional characteristics of the protective layer 18 described with reference to FIG. 3.

The sensor device 10 is thus formed.

The sensor device 10 thus formed may be further processed, for example, to form electrical contacts (wire bonding or solder balls or other) to electrically contact the electrical contact regions 9a, 9b and the contact 7, and/or to form further protection or passivation or packaging regions of the structure thus formed.

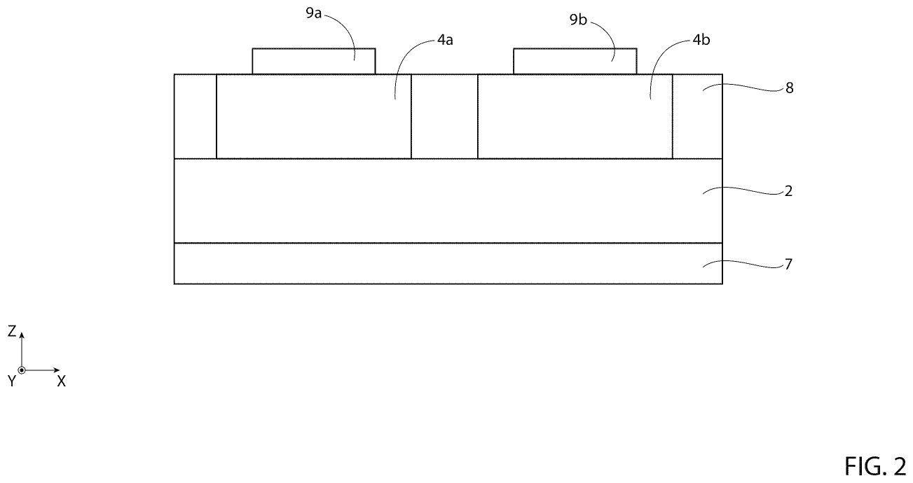

FIG. 2 illustrates, in the triaxial reference system of FIGS. 1A-1I, a further embodiment of the sensor device 10. In this embodiment, the insulation layer 8 that surrounds the sensor elements 4a, 4b (in particular extends between the sensor elements 4a, 4b) is made of the same material as the sensor layer 4, but undoped. Therefore, the insulation layer 8 is of undoped β-Ga₂O₃, which is electrically insulating, while the sensor layer 4 is made of doped β-Ga₂O₃, which is electrically semiconductive.

In this embodiment, the sensor layer 4 is deposited as described with reference to FIG. 1A, according to the embodiment that envisages the deposition of the sensor layer 4 in undoped form. Then, by a suitable implant mask (not illustrated), a localized doping of the β-Ga₂O₃ layer thus formed is performed exclusively at portions of the sensor layer 4 where it is desired to form the sensor elements 4a and 4b. In this manner, the portions of the sensor layer 4 protected by the implant mask during this doping proceeding remain undoped and therefore electrically insulating. Such undoped regions substantially correspond, in size, extension and shape, to the regions of the insulating layer 8 of FIG. 1F.

FIG. 3 illustrates, in the triaxial reference system of FIGS. 1A-1I, an embodiment of a gas sensor 20 integrated in the wafer or die 1.

The gas sensor 20 comprises the sensor device 10 of FIG. 1I or the sensor device 10 of FIG. 2.

The gas sensor 20 further comprises a protective layer 18, for example made of silicon nitride (SiₓN_{y}, e.g., Si₃N₄) that extends above and laterally to the electrical contact region 9b, completely covering the electrical contact region 9b and the sensor element 4b (if not completely covered by the electrical contact region 9b). The protective layer 18 completely insulates the electrical contact region 9b (and the sensor element 4b) from the external environment, providing protection from the gas(es), present in the external environment, to be sensed by the gas sensor 20. The electrical contact region 9a, conversely, is not protected and is therefore free to come into contact with such gas(es) to be sensed. In general, therefore, the material of the protective layer 18 is chosen in such a way as to be impermeable to the gas(es) to be sensed.

In a further embodiment, the electrical contact region 9a is also covered, in whole or in part, by a respective protection layer (not illustrated), of material chosen in such a way as to be permeable to the gas(es) to be sensed. In a further embodiment, the electrical contact region 9a is covered, only in part, by a respective protection layer (not illustrated), i. e,. such that this protection layer has openings configured to allow the flow of the gas to be sensed towards the electrical contact region 9a.

In one embodiment, the gas sensor 20 comprises, in an integrated form in the substrate 2 laterally and at a distance from the sensor device 10, a buried region 12, extending in the shape of a ring in plan view on the XY plane, surrounding in whole the sensor device 10 (closed ring) or surrounding in part the sensor device 10 (ring having an interruption of the shape). The buried region 12 has an electrical conductivity opposite with respect to the electrical conductivity of the substrate 2, and therefore in this embodiment it is of the P-type.

The buried region 12 is, for example, formed by implant of P-type doping species at the surface 2a of the substrate 2. The buried region 12 has, for example, a dopant concentration of the order of 5×10¹⁶ to 5×10¹⁷ at/cm³.

The buried region 12 directly faces the surface 2a of the substrate 2. The buried region 12 has, for example, a depth, along the direction of the Z-axis, comprised between 0.1 μm and 1 μm.

In one embodiment, the implant for the formation of the buried region 12 is performed prior to the formation of the sensor layer 4. In the embodiment wherein the implant for the formation of the buried region 12 is performed prior to the formation of the sensor layer 4, a first opening 15a and a second opening 15b are formed in the insulation layer 8 to electrically contact the buried region 12. The opening 15a extends laterally to the sensor element 4a while the opening 15b extends laterally to the sensor element 4b, such that the sensor elements 4a and 4b are interposed, along the X-axis, between the opening 15a and the opening 15b.

In a further embodiment not illustrated, the buried region 12 extends continuously between the opening 15a and the opening 15b below the sensor elements 4a, 4b.

In another embodiment, the implant for the formation of the buried region 12 is performed after the formation of the insulation layer 8, by forming an opening in the insulation layer 8. Said opening extends laterally to the sensor device 10, completely surrounding the sensor device 10.

In a further embodiment, the ring-shaped buried region 12 is replaced by two or more buried regions that extend, in view on the XY plane, as semicircles or strips or with a generic polygonal shape, surrounding in part the sensor device 10. Furthermore, it is evident that the annular shape of the buried region 12 previously described may be replaced by a corresponding polygonal shape.

In the embodiment where the openings 15a, 15b are present, the gas sensor 20 further comprises a first (metal) conductive region, which forms a first electrical contact 16, in the opening 15a, on the surface 2a of the substrate 2 and in electrical contact with the buried region 12; and the gas sensor 20 further comprises a second (metal) conductive region, which forms a second electrical contact 17 in the opening 15b, on the surface 2a of the substrate 2 and in electrical contact with the buried region 12.

In the embodiment wherein the implant for the formation of the buried region 12 is performed after the formation of the insulation layer 8, by opening an opening in the insulation layer 8, the first electrical contact 16 and the second electrical contact 17 are formed in said opening, such that the sensor elements 4a and 4b are interposed, along the X-axis, between the first electrical contact 16 and the second electrical contact 17.

Electrical connection elements, such as, for example, conductive wires (wire bonding), may be formed at the first and the second electrical contacts 16, 17, to bias them during use.

In use, by suitably biasing the first and the second electrical contacts 16, 17, an electric current flow may be generated through the buried region 12, thus generating heat by the Joule effect. A heater integrated in the substrate 2 is thus obtained, to heat the sensor elements 4a, 4b. For example, the voltage difference applied to the first and the second electrical contacts 16, 17 is comprised between 0.5 V and 5 V. In particular, the heater allows the two sensor elements 4a, 4b to be maintained at a same temperature.

Alternatively, a respective heater integrated in the substrate 2 (not shown) may be provided, as described above. Each heater is coupled to a respective sensor element 4a, 4b. Optionally, each heater is drivable to impose the same temperature to the two sensor elements 4a, 4b or different temperatures, as needed.

In a further embodiment, alternative to the formation of the heater integrated in the substrate 2, a heater may also be formed at the surface 2b of the substrate 2, at least in part aligned, along the Z-axis, with the sensor elements 4a, 4b, for example by depositing and patterning resistive material (e.g., in the shape of a serpentine line).

In general, a generic heater element may be coupled at the surface 2b of the substrate 2, below and aligned with the sensor elements 4a, 4b.

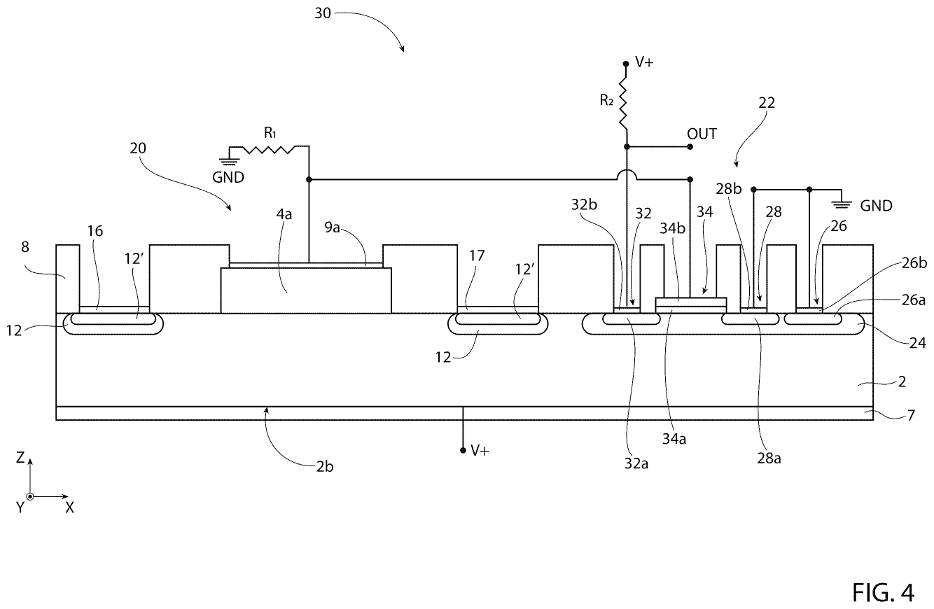

FIG. 4 illustrates, in the triaxial reference system of FIGS. 1A-1I, an embodiment of a gas sensor 30 integrated in the wafer or die 1, limitedly to some elements.

The gas sensor 30 comprises the gas sensor 20 of FIG. 3. In FIG. 4, the second sensor element 4b and the second conductive region 9b are not illustrated.

Optionally, the buried region 12 has the second electrical conductivity (P) and a first doping value and includes portions 12′ (having the second electrical conductivity P and a second doping value P+) integrated in the substrate 2, laterally and at a distance from the gas sensor 20.

The gas sensor 30 further comprises a transistor 22, extending at least in part in the substrate 2, laterally at a distance from the gas sensor 20. The transistor 22 includes a body region 24 extending in the substrate 2, directly facing the surface 2a of the substrate 2. The body region 24 has conductivity opposite to the conductivity of the substrate 2, and in this embodiment is of the P-type. The body region 24 has a dopant concentration comprised between 5×10¹⁶ and 1×10¹⁸ at/cm³.

The transistor 22 further includes a body terminal 26, a source terminal 28, a drain terminal 32, and a gate terminal 34. The body terminal 26, the source terminal 28, and the drain terminal 32 include respective doped regions 26a, 28a, and 32a, extending within the body region 24, facing the surface 2a of substrate 2, and exposed through respective openings in the insulating layer 8. The doped region 26a has an electrical conductivity of the same type as the body region 24, and has a doping species concentration comprised between 5×10¹⁸ and 1×10²⁰ at/cm³. The doped regions 28a and 32a have a conductivity opposite to the electrical conductivity of the body region 24, and have a doping species concentration comprised between 5×10¹⁸ and 2×10²⁰ at/cm³.

The body terminal 26, the source terminal 28 and the drain terminal 32 further include respective metal contacts 26b, 28b and 32b, on the surface 2a of the substrate 2, respectively in electrical contact with the doped regions 26a, 28a and 32a. The metal contacts 26b, 28b and 32b are, for example, made of Titanium (Ti), Nickel (Ni), Gold (Au), Aluminum (Al) or Copper (Cu).

The gate terminal 34 extends on the surface 2a of the substrate 2, in direct contact with the body region 24, interposed between the source terminal 28 and the drain terminal 32. The gate terminal 34 comprises a gate dielectric 34a, in contact with the surface 2a of the substrate 2, and a gate conductive region 34b, extending on the gate dielectric 34a. The gate dielectric 34a is, for example, made of silicon oxide (SiO₂) or aluminum oxide (Al₂O₃). The gate conductive region 34b is, for example, made of doped polysilicon or metal material.

The gate terminal 34 is electrically coupled to the first conductive region 9a. The gate terminal 34 and the first conductive region 9a are electrically coupled to a reference terminal which, in use, is capable of being biased to a reference potential GND through a first resistance R1. The drain terminal 32 is electrically coupled to a bias terminal which, during use, is capable of being biased to a bias voltage V+ through a second resistance R2. The body terminal 26 and the source terminal 28 are electrically coupled to the reference terminal so that, during use, they are biased to the reference potential GND. A further bias voltage applied, during use, to the substrate 2 through the electrical contact 7, may be comprised between GND and V+.

An output voltage or current of the gas sensor 30 is measured, in use, at a terminal OUT interposed between the second resistance R2 and the drain terminal 32 of the transistor 22.

In one embodiment, the first resistance R1 and the second resistance R2 are integrated in the substrate 2, for example they are provided by suitably doping respective regions in the substrate 2. In another embodiment, the first resistance R1 and the second resistance R2 are provided in the sensor layer 4, suitably doping respective portions of the sensor layer 4. In a further embodiment, the gas sensor 30 may include further circuit elements integrated in the substrate 2 in a manner known per se, such as, for example, diodes and capacitors.

In one embodiment, the gas sensor 30 includes a further transistor (not illustrated in FIG. 4) and two further resistances (not illustrated in FIG. 4), electrically coupled to the second sensor element 4b, in a manner similar to what has been described with reference to the transistor 22 and the resistances R₁ and R₂ electrically coupled to the first sensor element 4a.

In the sensor device 10, and similarly in the gas sensor 20 and the gas sensor 30, respective Schottky junctions are formed at the interface between the sensor elements 4a, 4b and the electrical contact regions 9a, 9b, when these are made of platinum. In a non-limiting example wherein the sensor device 10 (and similarly the gas sensor 20 or the gas sensor 30) is used for sensing hydrogen gas (H₂), the molecules of gas H₂ decompose at the surface of the electrical contact regions 9a, 9b, forming an electrically biased layer at the interface between the metal and the β-Ga₂O₃ of the sensor elements 4a, 4b. The electrically biased layer modifies the electric field in the device, modifying as a result the current-voltage (I-V) feature of the device.

In particular, in the gas sensor 30 of FIG. 4, during use, a variation in the electric field at the interface between the contact region 9a and the sensor element 4a causes a variation in the potential applied to the gate terminal 34 of the transistor 22, generating as a result a variation in the output voltage/current of the device measured at the terminal OUT.

In one embodiment wherein the gas sensor 20 includes the protective layer 18, the second sensor element 4b and the second contact region 9b are used, in use, as reference elements to carry out, in a manner known per se, differential measurements of the variation in output voltage/current. For example, in an embodiment of the gas sensor 30, an output current of the further transistor electrically coupled to the second sensor element 4b may be subtracted to an output current of the transistor 22, in a manner known per se.

The metal of the electrical contact regions 9a, 9b may catalyze the decomposition reaction of the gaseous molecules, and the efficiency of said reaction may increase as the temperature of the device increases.

Note that the use of crystalline β-Ga₂O₃ deposited through MOCVD technique on a 4H-SiC substrate ensures a high stability of the device at high temperatures (e.g., up to a temperature of 500°C.), ensuring as a result a high sensitivity in gas sensing. In particular, the 4H-SiC monocrystalline substrate allows the integration of heater elements as described with reference to the gas sensor 20 of FIG. 3, and of circuit elements such as, for example, transistors, resistances, diodes and capacitors as described with reference to the gas sensor 30 of FIG. 4.

Furthermore, the β-Ga₂O₃ of the sensor layer 4, deposited in the form of a crystalline film through MOCVD, provides, compared to non-crystalline films deposited, for example, in the form of inks or sol-gels, a higher sensitivity of the sensor due to a reduced leakage current. Furthermore, the sensor layer 4, being a crystalline film, may be used to integrate other functionalities, such as, for example, the resistances R1 and R2 described with reference to the gas sensor 30 of FIG. 4.

Finally, the β-Ga₂O₃ of the sensor layer 4 may be used in doped form as a semiconductor in the sensor elements 4a, 4b or in undoped form as an insulator, forming the insulating layer 8 as described with reference to FIG. 2. This allows semiconductive regions of β-Ga₂O₃ to be patterned through selective doping, without resorting to etch techniques.

1. A sensor device, comprising:
a substrate of 4H-SiC polytype silicon carbide having a first electrical conductivity type;
a first sensitive region of semiconductor material on a first side of the substrate; and
a first conductive region on and in electrical contact with the first sensitive region;
wherein the first sensitive region is made of β-polymorph crystalline gallium oxide (β-Ga₂O₃) having the first electrical conductivity type;
a second sensitive region on the first side of the substrate that is electrically isolated from the first sensitive region;
wherein the second sensitive region is made of β-Ga₂O₃ having the first electrical conductivity type;
a second conductive region on and in electrical contact with the second sensitive region; and
an insulating layer between the first and the second sensitive regions;
wherein the insulating layer is made of an insulating material selected from the group consisting of: silicon oxide, silicon nitride, TEOS, and undoped β-Ga₂O₃.

2. The sensor device according to claim 1, wherein the first and the second conductive regions are made of a metal material selected from the group consisting of: platinum, titanium, palladium, nickel, and tungsten.

3. The sensor device according to claim 1, further comprising a protective layer which completely covers the second sensitive region and the second conductive region.

4. The sensor device according to claim 1, further comprising a heater at least in part integrated in the substrate and thermally coupled to the first sensitive region.

5. The sensor device according to claim 1 configured as a gas sensor.

6. A semiconductor die, comprising:
the sensor device according to claim 1;
a transistor having a gate terminal, a first conduction terminal and a second conduction terminal, at least in part integrated in the substrate, the gate terminal being electrically coupled to the first conductive region;
a first resistance coupled between a first bias terminal and a first electrical node common to the gate terminal and the first conductive region; and
a second resistance coupled between a second bias terminal and one of the first and the second conduction terminals;
wherein the other of the first and the second conduction terminals is electrically coupled to the first bias terminal.

7. A method for manufacturing a sensor device, comprising the steps of:
forming, on a first side of a substrate of 4H-SiC polytype silicon carbide, having a first electrical conductivity type, a first sensitive region made of semiconductor material doped with the first electrical conductivity type; and
forming a first conductive region on and in electrical contact with the first sensitive region;
wherein the step of forming the first sensitive region comprises performing a Metal-Organic Chemical Vapor Deposition (MOCVD) growth process of β-polymorph crystalline gallium oxide (β-Ga₂O₃) having the first electrical conductivity type.

8. The method according to claim 7, further comprising the steps of:
forming a second sensitive region, having the first electrical conductivity type, on the first side of the substrate and electrically insulated from the first sensitive region;
wherein the step of forming the second sensitive region comprises performing the MOCVD growth process of β-Ga₂O₃; and
forming a second conductive region on and in electrical contact with the second sensitive region.

9. The method according to claim 8, wherein forming the first and the second sensitive regions comprises the steps of:
growing, by said MOCVD process, a sensitive layer of crystalline β-Ga₂O₃ having the first electrical conductivity type on said first side of the substrate; and
patterning the sensitive layer so as to form the first and the second sensitive regions at a physical distance from each other.

10. The method according to claim 9, wherein the step of electrically insulating the second sensitive region from the first sensitive region comprises forming an insulating layer between the first and the second sensitive regions.

11. The method according to claim 8, wherein forming the first and the second sensitive regions comprises the steps of:
growing, by said MOCVD process, a sensitive layer of undoped crystalline β-Ga₂O₃ on said side of the substrate; and
implanting, in respective spaced apart portions of said sensitive layer, doping species having the first electrical conductivity type to provide the first and the second sensitive regions.

12. The method according to claim 7, further comprising forming a heater at least in part integrated in the substrate and thermally coupled to the first sensitive region.

13. A sensor device, comprising:
a substrate of 4H-SiC polytype silicon carbide having a first electrical conductivity type;
a first sensitive region of semiconductor material on a first side of the substrate;
a second sensitive region on the first side of the substrate that is electrically isolated from the first sensitive region;
wherein the first and second sensitive regions are each made of β-polymorph crystalline gallium oxide (β-Ga₂O₃) having the first electrical conductivity type; and
an insulating layer between the first and the second sensitive regions;
wherein the insulating layer is made of undoped β-Ga₂O₃.

14. The sensor device according to claim 13, further comprising:
a first conductive region on and in electrical contact with the first sensitive region; and
a second conductive region on and in electrical contact with the second sensitive region.

15. The sensor device according to claim 14, wherein the first and the second conductive regions are made of a metal material.

16. The sensor device according to claim 14, further comprising a protective layer which completely covers over the second sensitive region.

17. The sensor device according to claim 14, further comprising a heater thermally coupled to the first sensitive region.
